(19) United States
(12) Patent Application Publication
(10) Pub. No.: US 2010/0010561 A1
(43) Pub. Date: Jan. 14, 2010

(54) BATTERY CHARGING STRUCTURE OF IMPLANTABLE PULSE GENERATOR AND PACEMAKER USING THE SAME

(76) Inventor: Choong Won Go, Seoul (KR)

Correspondence Address: PARK LAW FIRM, 3255 WILSHIRE BLVD, SUITE 1110, LOS ANGELES, CA, 90010, US

(21) Appl. No.: 12/526,739

(22) PCT Filed: Feb. 28, 2007
(86) PCT No.: PCT/KR07/01018

§ 371 Date: Aug. 11, 2009

(30) Foreign Application Priority Data
Feb. 12, 2007 (KR) 10-2007-0014507

(51) Int. Cl. A61N 1/375; H02J 7/00; A61N 1/378

(52) U.S. Cl. 607/36; 320/107

(57) ABSTRACT

A battery charging structure of an implantable pulse generator, by which the battery of the implantable generator can be charged so that the implantable pulse generator can be used permanently without replacing the implantable pulse generator, is disclosed.

### TECHNICAL FIELD

The present invention relates to a battery charging structure of implantable pulse generator and pacemaker using the same, and more specifically to a battery charging structure of implantable pulse generator, by which the battery of the implantable pulse generator can be charged for the implantable pulse generator to be used permanently without being replaced through surgery, high cost incurred by replacement surgery of the implantable pulse generator is reduced, and no side effects are produced, and a pacemaker using the same.

### BACKGROUND ART

In general, an implantable pulse generator implanted in the human body is a device for obtaining treatment effects by generating electric pulses by programmed control signals and transmitting these to the heart, nerves, or muscles, etc. to cause physiological reactions.

Such implantable pulse generators are classified largely into a pacemaker, a cerebral nerve stimulator, a vertebral nerve stimulator and a gastrointestinal nerve stimulator. Normally, such an implantable pulse generator consists of an electronic circuit for adjusting electric stimuli, a battery for supplying power to the electronic circuit, and a lead wire for transmitting the electricity generated from the electronic circuit to the human organ. The electric signals generated from the electronic circuit are transmitted to the human organ through the lead wire for the human organ to maintain normal function.

Especially, the pacemaker is the most widely used device of the aforementioned implantable pulse generator. It is used for a patient whose heart rate becomes abnormally slow so that blood going to the cerebrum is so insufficient to cause him vertigo or faint and whose heart output is decreased to cause him shortness of breath and heart failure. Namely, it is a device for helping him do activity or exercise that he was not able to do before because it was too hard for him to do, by maintaining the proper heart rate by sending a small quantity of electric stimuli to the heart when the heartbeat is not proper.

The pacemaker manufactured earlier for this purpose was too big and had many problems in terms of function. But recently as it became possible to make a small-sized pacemaker implantable in the human body with the advancement of electronic engineering, it is prolonging the life of many people and improving the quality of life as well.

Pacemakers implantable in the human body are classified according to its use into a VVI type (ventricular pacing, ventricular sensing, inhibited by ventricular events), a VVD type (ventricular pacing, dual chamber sensing, dual functioning), an AAI type (atrial pacing, atrial sensing, inhibited by atrial events), a DDD type (dual chamber pacing, dual chamber sensing, dual functioning), and an ICD type (implantable cardioverter defibrillator). The term “pacemaker” used in the present invention generally refers to the aforementioned pacemaker regardless of its use or shape.

As shown in FIG. 1, a conventional pacemaker generally comprises a pacemaker 10, a lead wire 20 and an electrode 30. Specifically, the pacemaker 10 consists of an electronic circuit for adjusting electric stimuli and a battery for supplying power to the electronic circuit that are built in a case 11 made usually of metal. The lead wire 20 plays the role of transmitting to the heart the electricity generated from the pacemaker 10 to beat the heart or sending the electric signals generated from the heart to the pacemaker 10. The electrode 30 provided at the end portion of the lead wire 20 transmits to the heart muscle the electric stimuli transmitted through the lead wire 20 for the heart to maintain a proper number of heart beats.

However, the conventional pacemaker has a problem that the battery life is 5 to 7 years, so the pacemaker should be replaced through surgery when the battery life has run out. Namely, assuming a 50 year old male patient lives as long as the average lifetime of 80 years, he has to undergo several surgeries so as to replace about 5 to 8 pacemakers, so he is burdened with high surgery costs for replacing the pacemaker. Moreover, for surgery of replacing the pacemaker, the skin is cut open, so the problem is that not only the surgery process is complicated and takes a long time for recovery but also it could cause harmful side effects after surgery.

### DISCLOSURE OF INVENTION

### Technical Problem

The present invention is to solve the above-mentioned problems with an object to provide a battery charging structure of an implantable pulse generator which can be used permanently without replacing the implantable pulse generator through surgery because the battery of the implantable pulse generator can be charged, and eliminates high cost for replacement surgery of the implantable pulse generator and causes no harmful side effects.

It is another object of the present invention to provide a pacemaker that adopts the charging structure of the implantable pulse generator.

### Technical Solution

In accordance with one aspect the present invention, there is provided a battery charging structure of an implantable pulse generator which has an electronic circuit built in a case and a battery for supplying power to the electronic circuit, and transmits the electric signals generated from the electronic circuit to the human organ through a lead wire, the battery charging structure characterized in that: said battery is constructed as a chargeable battery; and said chargeable battery is constructed to be charged with electricity by being grounded in such a way that a needle-shaped charging needle with power applied from a power supply unit is inserted.

In accordance with another aspect the present invention, there is provided a pacemaker having an electronic circuit built in a case and a battery for supplying power to the electronic circuit, the pacemaker characterized in that: said battery is constructed as a chargeable battery; a charging hole is formed in said case, and the inside of the charging hole is provided with battery charging terminals electrically connected to said chargeable battery; and a needle-shaped charging needle is inserted into said charging hole, and the needle charging terminals provided at the end portion of the charging needle are divided into two by an insulator, so that after the charging needle is inserted into the charging hole the needle charging terminals contact with the corresponding battery charging terminals so as to charge the battery, and the pacemaker further comprises a power supply unit which is electrically connected to said needle charging terminals to apply power to said needle charging terminals.

### ADVANTAGEOUS EFFECTS

According to the battery charging structure of implantable pulse generator and pacemaker using the same of the present invention, it has the effects that it is possible to use the implantable pulse generator without replacing it through surgery because the battery of the implantable pulse generator can be charged, and high cost incurred by the replacement surgery of the implantable pulse generator is eliminated and no side effects are produced.

### BRIEF DESCRIPTION OF THE DRAWINGS

These and other features, aspects, and advantages of preferred embodiments of the present invention will be more fully described in the following detailed description, taken in conjunction with the accompanying drawings. In the drawings:

FIG. 1 is a schematic view illustrating how the conventional pacemaker is fitted in the human body;

FIG. 2 is a perspective view to which a battery charging structure of implantable pulse generator according to the present invention is applied;

FIG. 3 is a sectional view to which the battery charging structure of implantable pulse generator according to the present invention is applied;

FIG. 4 is a sectional view of another embodiment of a battery charging terminal according to the present invention;

FIG. 5 is a sectional view of a pacemaker according to another embodiment of the present invention; and

FIGS. 6 to 9 are drawings for describing the battery charging method of the pacemaker according to the present invention.

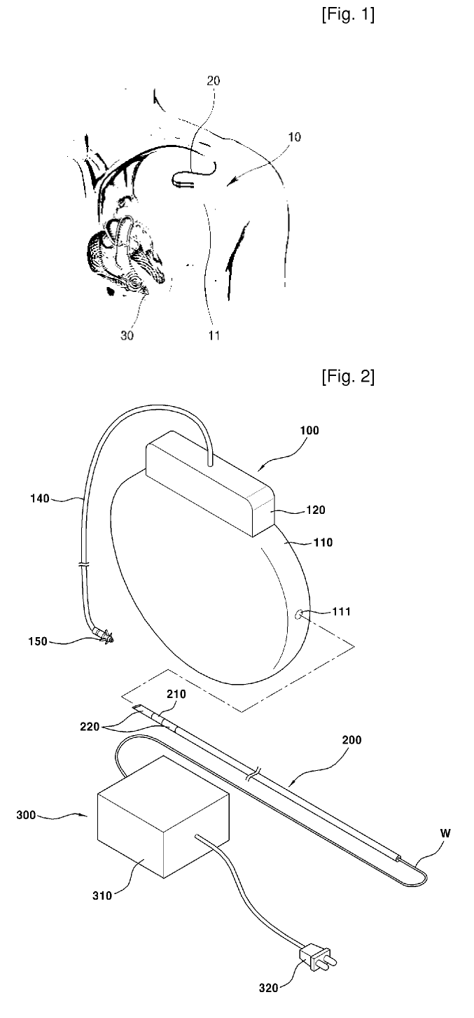

### DESCRIPTION OF NUMERALS FOR MAIN PORTIONS OF THE DRAWINGS

100: pacemaker, 110: case, 111: charging hole, 111a: plug, 111b: guide stopper, 120: electronic circuit, 130: battery, 131: battery charging terminal, 140: lead wire, 150: electrode, 200: charging needle, 210: insulator, 220: needle charging terminal, 300: power supply unit, 310: converter, 320: power source, W: wire

### BEST MODE FOR CARRYING OUT THE INVENTION

Below will be described in detail a battery charging structure of implantable pulse generator and pacemaker using the same of the present invention with reference to the accompanying drawings.

The battery charging structure of implantable pulse generator according to the present invention has an electronic circuit and a battery for supplying power to the electronic circuit built in a case, and electric signals generated from the electronic circuit are transmitted to the human organ through the lead wire. In accordance with the implantable pulse generator, the battery consists of a chargeable battery, which is grounded in such a way that needle-shaped charging needles having power applied thereto are inserted for charging to be achieved.

For this end, a charging hole is formed in the case, and the inside of the charging hole is provided with battery charging terminals electrically connected to the battery and the charging hole is constructed for the needle-shaped charging needle to be inserted. And, the needle charging terminals provided at the end portion of the charging needle are divided into two by an insulator, so the needle charging terminals are constructed for the battery to be charged by contact with the corresponding battery charging terminals after the charging needle is inserted into the charging hole.

In addition, the needle charging terminals are constructed so as to have power applied from the power supply unit to which they are electrically connected.

Here, the implantable pulse generator according to the present invention, as described above, comprises all the devices such as a pacemaker, a cerebral nerve stimulator, a vertebral nerve stimulator and a gastrointestinal nerve stimulator, which are implanted in the human body and transmit electrical signals generated from the electronic circuit to the human organs through a lead wire for the human organs to maintain normal functions.

The battery charging structure of implantable pulse generator according to the present invention constructed as described above can charge the battery by inserting the charging needle into the charging hole of the case provided with the battery charging terminals when the battery is discharged, so the implantable pulse generator can be used permanently without being replaced through surgery.

### Mode for the Invention

Below will be described a pacemaker, an example of a charging structure of implantable pulse generator according to the present invention, in more detail with reference to the accompanying drawings.

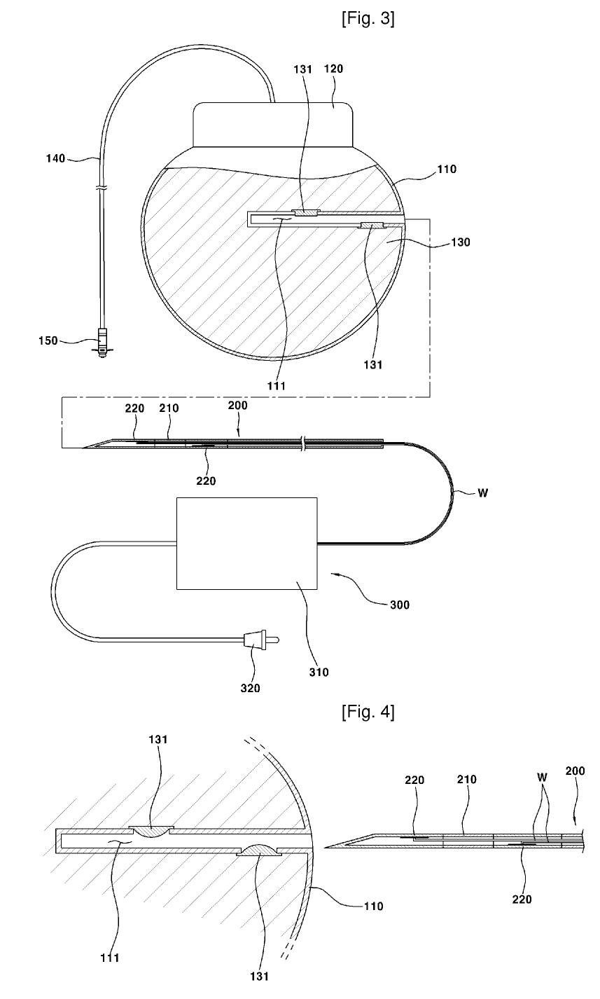

The accompanying FIG. 2 is a perspective view of a pacemaker to which the charging structure of implantable pulse generator according to the present invention is applied, and FIG. 3 is a sectional view of the pacemaker to which the charging structure of implantable pulse generator according to the present invention is applied.

The pacemaker according to the present invention, unlike the conventional pacemaker which is replaced through surgery when the battery life has run out, is constructed in such a way that the battery of the pacemaker can be charged. Therefore, it is characterized in that the pacemaker can be used permanently, and furthermore, no high cost is incurred by replacement surgery of the pacemaker, and no side effects are produced.

Referring to the accompanying drawings, the pacemaker according to the present invention comprises a pacemaker 100, a charging needle 200 and a power supply unit 300.

The pacemaker 100, similar to the conventional structure, includes an electronic circuit 120 for adjusting electric stimuli and a battery 130 for supplying power to the electronic circuit built in a case 110. Of course, the pacemaker 100 is provided with a lead wire 140 which transmits electricity generated from the electronic circuit 120 to the heart to beat it or sends electric signals generated from the heart to the electronic circuit 120. The end portion of the lead wire 120 is provided with an electrode 150 which transmits to the heart muscle the electric stimuli transmitted through the lead wire 120 in direct contact with the muscle of the heart for the heart to maintain a proper heart rate.

Here, the pacemaker 100 according to the present invention has the battery constructed as a chargeable battery 130 to make charging possible and a charging hole 111 formed in the case 110. And the inside of the charging hole 111 is provided with conductive battery charging terminals 131 for receiving the current supplied from a charging needle 200, which is to be described later, and charging the battery 130. Normally, since the thickness of a pacemaker is made thin, it is preferable to form the charging hole 111 starting from the side of the case 110 and extended to a given depth in the direction of the center of the battery 130.

The charging needle 200 made in a needle shape of a given depth is inserted into the charging hole 111. Here, since the charging needle 200 having a given elasticity is formed with the inside being in a form of empty space into which the lead wire is inserted, it is preferable to electrically connect the charging needle by wire with the power supply unit, which is to be described later. The length of the charging needle 200 may be constructed to a suitable length so as to be inserted into the charging hole 111 of the pacemaker installed in the inside of the human body from the outside of the human body.

Here, the needle charging terminals 220 provided at the end portion of the charging needle 200, that is, the end portion inserted into the charging hole 111, are divided into two so as to obtain the corresponding battery charging terminals by the insulator 210 in between. Accordingly, it is constructed in such a way that when the end portion of the charging needle 200 is inserted into the charging hole 111, the needle charging terminals 220 contact with the corresponding battery charging terminals 131. Namely, the end portion of the charging needle 200 is divided into two needle charging terminals 220 with one side for positive (+) pole and the other side for negative (−) pole by the insulator 210 in between, so when power is supplied while the corresponding battery charging terminals 131 are in contact, the battery is charged by electricity supplied from power source.

Meanwhile, the battery charging terminals 131 may be constructed in a convex arch form, as shown in FIG. 4. If the charging structure is constructed in such a form in which the needle charging terminals 220 are combined to the battery charging terminals shaped like a convex arch, the contact force between charging terminals 131 and 220 is significantly improved so as to make charging stable.

The power supply unit 300 is electrically connected with the needle charging terminals 220 to apply electricity from power source to the needle charging terminals 220. Namely, as described above, the needle charging terminals 220 are electrically connected with the power supply unit by the wire W arranged in the inside of the charging needle 200 so that the battery 130 is charged by electricity supplied from the power source through the needle charging terminals 220. Here, the power supply unit 300 may use various means in public domain such as chargeable battery, but it is preferable, as shown, to construct it with a converter 310 for converting AC supplied from a power source 320 into DC to be supplied to the needle charging terminals 220. That is, the converter 310 has AC of 90 V to 250 V supplied from power source 320 to supply DC necessary for charging the battery 130. Here, though not shown, it is preferable that the converter 310 provides a trip circuit for shutting off power supply automatically when charging of the battery 130 is completed.

And, as shown in FIG. 5, it is preferable that a plug 111a having an elastic restoring force is installed at the entrance of the charging hole 111 to seal the inside of the charging hole 111. Here, it is preferable that the plug 111a is made of rubber or insulating silicon material which has excellent restoring force but harmless to the human body and assemble and fix it at the entrance of the charging hole 111. Like this by sealing the charging hole 111 with the plug 111a, it is possible to prevent human secretion from entering the charging hole 111. Also when the charging needle 200 is inserted into the charging hole 111 for battery charging, if the charging needle 200 is inserted through the plug 111a inserted at the entrance of the charging hole 111, the plug is adhered to while embracing the outer circumference of the charging needle 200 by the elastic force of the plug itself, so it prevents secretion from entering the charging hole 111. Also, even if the charging needle 200 is separated from the charging hole 111 after charging of the battery 130 is completed, the plug 111a whose restoring force is excellent seals the charging hole 111 while contracting again by the elastic force of the plug itself, so it prevents secretion from entering the charging hole 111.

Also, as shown in FIG. 5, it is preferable to form the entrance of the charging hole 111 sloped and form a guide stopper 111b upward and downward of the case on one side of the charging hole entrance. Namely, the end portion of the charging needle 200 inserted in the human body to charge the battery 130 of the pacemaker slides naturally toward the entrance of the charging hole 111 by being caught on the guide stopper 111b formed at the entrance of the charging hole 111, while the entrance of the charging hole is formed sloped so as to widen the area of the entrance of the charging hole 111, so the charging needle 200 can be inserted easily into the charging hole 111.

The charging method of the pacemaker according to the present invention constructed as described above will be described with reference to FIGS. 6 to 9. As shown in FIG. 6, after the location into which the charging needle 200 is to be inserted is cleaned with sterilizer and local anesthesia is carried out, the charging needle 200 is inserted toward the charging hole 111 of the pacemaker. At this time, the charging needle 200 is preferably inserted from the side of the human body M corresponding to the charging hole 111 formed on the side of the pacemaker 100. Especially, it is preferable to perform surgery while confirming the accurate location of insertion of the charging needle 200 by using radioactive image projector.

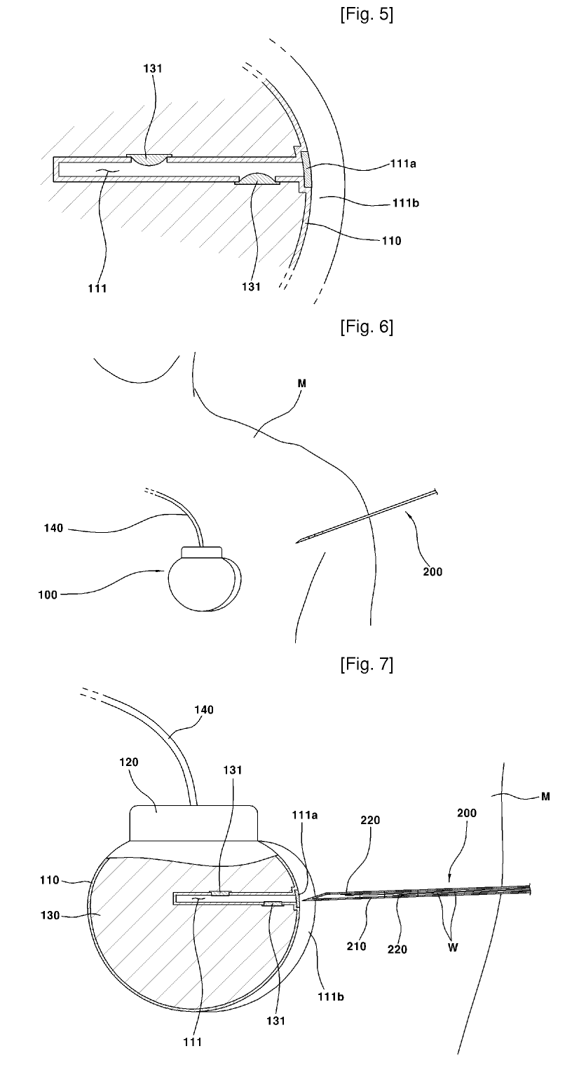

And as shown in FIG. 7, when the end portion of the charging needle 200 is located at the entrance of the charging hole 111, the end portion of the charging needle 200 inserted in the human body M, caught by the guide stopper 111b formed at the entrance of the charging hole 111, is naturally inserted sliding into the entrance of the charging hole while passing through the plug 111a installed at the entrance of the charging hole 111.

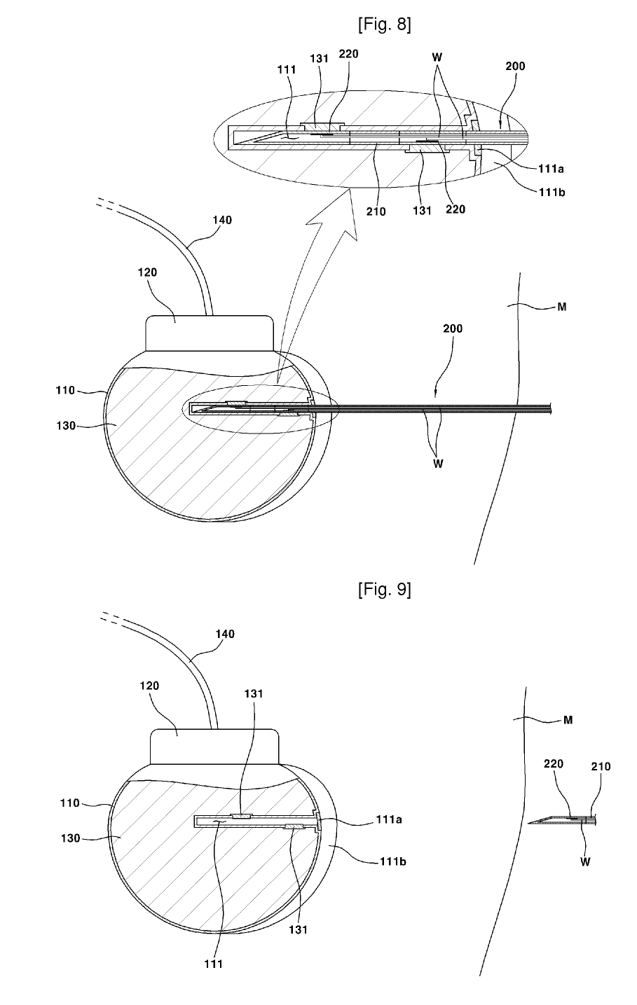

Then, as shown in FIG. 8, the end portion of the charging needle 200 is inserted into the charging hole 111, and the needle charging terminals 220 provided at the end portion of the charging needle 200 adhere to the corresponding battery charging terminals 131 provided in the charging hole 111. At this time, the plug 111a installed at the entrance of the charging hole 111 adheres to the outer circumference of the charging needle 200 that has passed therethrough by elastic force of the plug itself to prevent secretion from entering the charging hole 111. After that, when the converter 310 is turned on, AC supplied from power source is converted to DC to be applied to the charging terminals 220, and through the battery charging terminals 131 in contact with the needle charging terminals 220, current is supplied to the battery 130 to charge it.

After that, as shown in FIG. 9, when charging of the battery 130 is completed, the charging needle 200 is separated from the charging hole 111 and the charging needle 200 is pulled out of the human body M, to complete charging surgery according to the present invention. At this time, even though the charging needle 200 is separated from the charging hole 111, the plug 111a with excellent restoring force installed at the entrance of the charging hole 111 is again contracted by the elastic force of the plug itself to seal the charging hole 111, so secretion is prevented from entering the charging hole 111.

### INDUSTRIAL APPLICABILITY

As described above, the pacemaker according to the present invention has an advantage that it is not necessary to conventionally replace the pacemaker whose battery life has run out, and when the battery of the pacemaker is discharged, the battery can be charged for continuous use of the pacemaker, so high cost is not incurred by replacement of the pacemaker.

Another advantage is that unlike the conventional method of cutting open the skin to replace the pacemaker, the battery of the pacemaker is charged by inserting a charging needle, so the region of surgery is too small to make a scar and the surgery process is very simple, and also daily life is possible without side effects right after charging of the pacemaker which uses the battery.

Although the present invention has been described in connection with the exemplary embodiments illustrated in the drawings, it is only illustrative. It will be understood by those skilled in the art that various modifications and equivalents can be made to the present invention. Therefore, the true technical scope of the present invention should be defined by the appended claims.

1. A battery charging structure of an implantable pulse generator which has an electronic circuit built in a case and a battery for supplying power to the electronic circuit, and transmits the electric signals generated from the electronic circuit to the human organ through a lead wire, the battery charging structure characterized in that:
said battery is constructed as a chargeable battery; and
said chargeable battery is constructed to be charged with electricity by being grounded in such a way that a needle-shaped charging needle with power applied from a power supply unit is inserted.

2. The battery charging structure of claim 1, characterized in that a charging hole is formed in the case that has said chargeable battery built in and the inside of the charging hole is provided with battery charging terminals electrically connected to said chargeable battery; and
a needle-shaped charging needle is inserted in said charging hole, and the needle charging terminals provided at the end portion of the charging needle are divided into two by an insulator so that after the charging needle is inserted into the charging hole the needle charging terminals contact with the corresponding battery charging terminals so as to charge the battery.

3. A pacemaker having an electronic circuit built in a case and a battery for supplying power to the electronic circuit, the pacemaker characterized in that:
said battery is constructed as a chargeable battery;
a charging hole is formed in said case, and the inside of the charging hole is provided with battery charging terminals electrically connected to said chargeable battery; and
a needle-shaped charging needle is inserted into said charging hole, and the needle charging terminals provided at the end portion of the charging needle are divided into two by an insulator, so that after the charging needle is inserted into the charging hole the needle charging terminals contact with the corresponding battery charging terminals so as to charge the battery, and
the pacemaker further comprises a power supply unit which is electrically connected to said needle charging terminals to apply power to said needle charging terminals.

4. The pacemaker of claim 3, characterized in that a plug having restoring force is installed at the entrance of said charging hole to seal the inside of said charging hole.

5. The pacemaker of claim 3, characterized in that the entrance of said charging hole is formed sloped to make it easy for said charging needle to enter into said charging hole, and a guide stopper is formed upward and downward of the case on one side of the entrance of said charging hole.
